(19) United States
(12) Patent Application Publication
(10) Pub. No.: US 2012/0058497 A1
(43) Pub. Date: Mar. 8, 2012

(54) DIAGNOSTIC REAGENT FOR CROHN'S DISEASE

(75) Inventors: Yasuyo SUGA, Kawasaki-shi (JP); Maiko MORI, Kawasaki-shi (JP); Masaki HASHIMOTO, Kawasaki-shi (JP); Manabu SUZUKI, Kawasaki-shi (JP); Tomomi MORIGUCHI, Kawasaki-shi (JP); Takaaki KAWAGUCHI, Shinjuku-ku (JP); Keiko SAITO, Shinjuku-ku (JP); Masakazu TAKAZOE, Shinjuku-ku (JP)

(73) Assignee: AJINOMOTO CO., INC., Tokyo (JP)

(21) Appl. No.: 13/225,087

(22) Filed: Sep. 2, 2011

Related U.S. Application Data
(63) Continuation of application No. PCT/JP10/53682, filed on Mar. 5, 2010.

(30) Foreign Application Priority Data
Mar. 5, 2009 (JP) 2009-052692

(51) Int. Cl. G01N 33/566; C07K 14/415

(52) U.S. Cl. 435/7.92; 530/370; 530/372; 530/379

(57) ABSTRACT

The present invention provides a reagent and a method of safely, conveniently, and specifically diagnosing Crohn's disease. Provided are a diagnostic method for Crohn's disease in a subject, including measuring antibodies against one kind or more of dietary components selected from the group consisting of grapefruit, alfalfa, avocado, cabbage, green pepper, lettuce, onion, potato (white), spinach, tomato, oat, pecan, yeast, cane sugar, celery, buckwheat, corn, rice and soy, and a diagnostic reagent or kit for Crohn's disease, containing the above-mentioned preparation of a dietary component.

### TECHNICAL FIELD

The present invention relates to a method of specifically diagnosing Crohn's disease safely and highly sensitively, a reagent for diagnosing Crohn's disease, and a diagnostic kit comprising the reagent.

### BACKGROUND ART

Crohn's disease, like ulcerative colitis (UC), is a disease categorized under local inflammatory bowel diseases (IBDs) based on abnormalities of immune responses. Unlike UC, in which inflammation is localized to the large intestine, Crohn's disease is a disease characterized by ulceration at possibly all sites of the digestive tract, from the mouth to the anus. Major clinical symptoms are abdominal pain and diarrhea, often accompanied by further symptoms such as fever, melena, weight loss due to malabsorption, general malaise, and anemia. The disease is sometimes complicated by extraintestinal complications, including skin symptoms such as pyoderma gangrenosum and erythema nodosum, joint lesions, stomatitis and the like.

However, the etiology of Crohn's disease has not yet been clarified well, the diagnosis and treatment thereof being subject to limitations.

For example, the diagnosis of Crohn's disease has been made on the basis of a comprehensive assessment of clinical findings, as well as radiographic examination, endoscopic examination, histologic examination of endoscopically collected biopsy specimens, and the like; however, these examinations involve much time to clean the inside of the gut before the exam and pose great burdens, both physically and mentally, on the patient, including administration of contrast medium and insertion of an endoscope into the gut, and in addition require much experience and skilled art for technical procedures and differential judgement.

In the case of the small intestine type or the small intestine dominant small intestine-large intestine type, in particular, radiographic examination or endoscopic examination requires still higher technical levels for the reasons of the structural complexity of the small intestine, the remoteness of the site of onset from the site where the endoscope is inserted (anus or mouth) and the like.

Also, there are many cases in which the disease is difficult to distinguish from other inflammatory bowel diseases that exhibit similar pathological findings, such as UC, and there is a demand for a diagnostic method that is safe, convenient, and specific for Crohn's disease.

It is thought that a wide variety of genetic and environmental factors complexly influence the onset and exacerbation of Crohn's disease; in particular, the involvement of antigens derived from microorganisms, including enteric bacteria, has been suggested; abnormal immune responses to microorganisms are thought to be profoundly associated with inflammatory reactions in Crohn's disease. For example, it has been reported that anti-Saccharomyces cerevisiae antibody (ASCA), anti-I2 antibody, anti-outer membrane protein C (OmpC) antibody, anti-flagellin antibody and the like increase specifically in the sera of Crohn's disease patients. Meanwhile, besides microorganisms, reports are available that antibody titers against CRP and swine amylase increase specifically in the sera of Crohn's disease patients. Based on these findings, noninvasive diagnostic methods for Crohn's disease by detection of these antibodies have been proposed (patent documents 1 and 2, non-patent documents 1 to 3).

However, in all these methods with the use of a single antibody as a marker, the sensitivity (true positive rate) is at most about 40%. Although the sensitivity can be improved by combining a plurality of antibodies (patent document 3), 12 antigen, OmpC antigen, flagellin antigen, and CRP antigen are problematic with difficulty in their obtainment and the like.

Diets, like enteric bacteria, can influence intestinal immunity as a foreign substance that passes the gut. Because elemental diet therapy is effective in the treatment of Crohn's disease, the presence of some dietary antigens as an etiology or exacerbating factor is suggested; however, the involvement of any particular dietary antigen in Crohn's disease has not yet been clarified.

### DOCUMENT LIST

### Patent Documents

patent document 1: JP-A-H11-190734
patent document 2: JP-A-2004-526122
patent document 3: JP-A-2006-308494

### Non-Patent Documents

non-patent document 1: Jpn J Electroph 1999; 43:139-145
non-patent document 2: Gastroenterology 2002; 123:689-699
non-patent document 3: Gastroenterology 2000; 119:23-31

### SUMMARY OF THE INVENTION

### Problems to be Solved by the Invention

A problem to be solved by the present invention is to provide a novel method of safely, conveniently, and specifically diagnosing Crohn's disease, a reagent therefor, and a diagnostic kit comprising the same.

### Means of Solving the Problems

In solving the above-described problem, the present inventors first took note of a report that patients with Crohn's disease are likely to be exposed to antigens because of enhanced membrane permeability due to a disorder of the barrier mechanism of intestinal epithelium, and conceived that dietary antigens may be involved in inflammatory reactions in Crohn's disease. Hence, the present inventors measured and compared antibody titers against various dietary components in sera collected from Crohn's disease patients, UC patients and healthy persons. As a result, the present inventors succeeded in identifying 19 items of dietary components that exhibited specifically elevated serum antibody titers in specimens from Crohn's disease patients. Thereof, 14 items were found to be dietary components that have been not yet been suggested as being associated with Crohn's disease at all.

Furthermore, the present inventors confirmed that by using in combination two kinds or more out of these 19 items, the sensitivity and specificity (true negative rate) of the diagnosis of Crohn's disease can be further improved, and have developed the present invention.

Accordingly, the present invention is as follows:

[1] A diagnostic method for Crohn's disease in a subject, comprising measuring antibodies against one kind or more of dietary components selected from the group consisting of grapefruit, alfalfa, avocado, cabbage, green pepper, lettuce, onion, potato (white), spinach, tomato, oat, pecan, yeast, cane sugar, celery, buckwheat, corn, rice and soy (with the provision that when measuring an antibody against one kind of dietary component, the dietary component is other than celery, buckwheat, corn, yeast and soy) in a specimen collected from the subject.
[2] The method according to [1], wherein the method comprises measuring antibodies against two kinds or more of dietary components.
[3] The method according to [2], wherein at least one kind of dietary component is other than celery, buckwheat, corn, yeast and soy.
[4] The method according to [2] or [3], wherein the method comprises measuring an antibody against at least one kind of dietary component selected from the group consisting of grapefruit, cabbage, lettuce, oat, pecan, yeast, cane sugar, celery, buckwheat and corn.
[5] The method according to any one of [2] to [4], wherein the method comprises measuring antibodies against at least yeast and corn.
[6] The method according to any one of [2] to [5], wherein the method comprises measuring antibodies against three kinds or more of dietary components.
[7] The method according to [6], wherein the method comprises measuring antibodies against at least yeast, corn, and buckwheat or celery.
[8] The method according to any one of [1] to [7], wherein the method comprises measuring antibodies against polypeptide antigens contained in dietary components.
[9] The method according to [8], wherein the polypeptide antigen is Glutelin.
[10] A reagent for diagnosis of Crohn's disease comprising a preparation of a dietary component selected from the group consisting of grapefruit, alfalfa, avocado, cabbage, green pepper, lettuce, onion, potato (white), spinach, tomato, oat, pecan, rice and cane sugar.
[11] The reagent according to [10], wherein the preparation is an isolated or purified polypeptide antigen.
[12] The reagent according to [11], wherein the polypeptide antigen is Glutelin or a partial peptide thereof possessing antigenicity.
[13] A kit for diagnosis of Crohn's disease comprising a preparation of two kinds or more of dietary components selected from the group consisting of grapefruit, alfalfa, avocado, cabbage, green pepper, lettuce, onion, potato (white), spinach, tomato, oat, pecan, yeast, cane sugar, celery, buckwheat, corn, rice and soy.
[14] The kit according to [13], wherein the preparation is an isolated or purified polypeptide antigen.
[15] The kit according to [14], wherein the polypeptide antigen is Glutelin or a partial peptide thereof possessing antigenicity.

### EFFECT OF THE INVENTION

The diagnostic method of the present invention is remarkably effective in dramatically improving the sensitivity and specificity of the diagnosis of Crohn's disease and narrowing the application range of the diagnosis by invasive methods such as endoscopy, which require high technical skills and pose major burdens on the patient, by combining determinations of antibody titers of Crohn's disease-specific anti-dietary-component antibodies. Another advantage is that reagents are easily available because of the use of dietary components.

### BRIEF DESCRIPTION OF THE DRAWINGS

FIG. 1 shows results of CBB staining of rice proteins. Lane M shows the results for a protein molecular weight marker; lane “a” shows the results for rice proteins; the numerical values on the left end indicate the molecular weights (kDa) of the proteins.

FIG. 2 shows results of Western blotting of rice proteins. Lane C shows the results obtained using a serum from a Crohn's disease patient (CD) as the antibody; lane H shows the results obtained using a serum from a healthy volunteer (HC) as the antibody; the numerical values on the left end indicate the molecular weights (kDa) of the proteins. “A” indicates a band that exhibited a specific reaction to the antibody in the serum from the CD.

### MODES FOR CARRYING OUT THE INVENTION

The present invention provides a diagnostic method for Crohn's disease comprising measuring one kind or more of Crohn's disease-specific anti-dietary-component antibodies in a specimen collected from a subject of diagnosis. In the present invention, “a Crohn's disease-specific anti-dietary-component antibody” refers to an antibody against a dietary component, which the antibody titer rises specifically in animals suffering Crohn's disease, specifically meaning an antibody against one of 19 kinds of dietary components: grapefruit, alfalfa, avocado, cabbage, green pepper, lettuce, onion, potato (white), spinach, tomato, oat, pecan, yeast, cane sugar, celery, buckwheat, corn, rice and soy (hereinafter also referred to as “an anti-dietary-component antibody of the present invention”). Here, “Crohn's disease-specific” refers to a state wherein the frequency of elevation of antibody titer (positive rate; true positive rate) in animals suffering Crohn's disease is significantly higher than the frequency in animals suffering at least UC and healthy animals.

An anti-dietary-component antibody of the present invention may be an antibody against a polypeptide antigen contained in various dietary components (antigen of each dietary component). The polypeptide antigen of the dietary component may specifically be any polypeptide antigen contained in a dietary component of grapefruit, alfalfa, avocado, cabbage, green pepper, lettuce, onion, potato (white), spinach, tomato, oat, pecan, yeast, cane sugar, celery, buckwheat, corn, rice or soy, and specific for Crohn's disease. In the present invention, out of the aforementioned 19 kinds of dietary components, rice, buckwheat, corn and cane sugar are preferable; polypeptide antigens contained therein include glutelin, glyoxylase 1, enolase, UDP-glucose pyrophosphorylase, asparatic protease, prolamin, oleosin and the like in the case of rice, clathrin and the like in the case of buckwheat, 2,3-bisphosphoglycerate-independent phosphoglyceratemutase, protein disulfide isomerase, ketol-acid reductoisomerase, elongation factor 1 alpha, phenylalanine ammonia-lyase and the like in the case of corn, and triphosphate isomerase 1, NBS-LRR type RGA and the like in the case of cane sugar.

In the diagnostic method of the present invention, the subject of diagnosis may be an optionally chosen animal that can contract Crohn's disease (including non-human inflammatory bowel diseases corresponding to human Crohn's disease), and is, for example, a mammal, specifically exemplified by humans, non-human primates, dogs, cats, rabbits, rats, mice and the like.

The specimen for the diagnosis is not particularly limited, as far as it is a component or tissue that can be isolated from a subject of diagnosis and is derived from the subject in which a Crohn's disease-specific anti-dietary-component antibody is possibly present; examples include blood (whole blood, serum, plasma), saliva, other humoral fluids, various tissues and the like, with preference given to serum or plasma. When using a specimen from an ulcerative colitis patient for reference and/or from a healthy person for control, the same applies to these specimens.

The method of measuring each antibody in the specimen may be any method for use to detect and measure an antibody as an immunological assay; although any commonly used measuring method with an enzyme, fluorescent substance, luminescent substance, radioactive substance, coloring substance or the like as the labeling substance can be used, an enzyme immunoassay method, an immunochromatography method and the like are preferred, with greater preference given to an enzyme immunoassay method, for example, an ELISA method, because the amount of antibody in the specimen can be easily numerated by color intensity or absorbance. For simultaneously measuring antibodies against multiple items, commercially available ELISA kits enabling measurements of IgG in 93 food items (93 Food IgGScreen/GENESIS Diagnostics Company and the like), for example, can be used, or measurements may be outsourced to private testing organizations (IgG Food Antibody Assessment/Genova Diagnostics Company and the like). Various instruments, supplies, reagents, labeling methods and measuring conditions in public knowledge can be used as required for the measurement.

For measuring the amount of label bound to the antibody, a commonly used method is chosen according to the kind of labeling substance used; for example, when using an enzyme as the labeling substance, it is advantageous to measure the decomposition of a color-developing substrate as absorbance using a spectrophotometer.

For example, a measurement of an anti-dietary-component antibody of the present invention by an ELISA method can be achieved by first reacting a specimen with a dietary component preparation to form an antigen-antibody complex, then adding an enzyme-labeled substance that reacts with the anti-dietary-component antibody, further adding an enzyme substrate to allow the reaction to proceed, and measuring the amount of the label on the reaction product by enzyme activity.

A dietary component preparation can be prepared by processing a dietary component by a commonly used method. Dietary components used as a material include commercially available fresh, frozen, or freeze-dried products, and powder materials thereof (Allergon Company and the like); the method of processing a dietary component is preferably extraction. The extraction can be achieved by, for example, processing the material physically (sonication, French press, mortar, homogenizer, glass beads, freeze-thawing and the like), or using a surfactant, in water, an organic solvent, a buffer solution, or glycerol. It is possible to raise the extraction efficiency by adding a salt or by warming. Useful dietary component preparations include, in addition to extracts obtained by the above-described method, commercially available protein extracts (BioChain Company, Antigen Laboratories Company and the like) and/or allergen extracts for diagnosis (Torii Pharmaceutical Co., Ltd., Antigen Laboratories Company and the like) and the like.

The dietary component preparation may be an isolated or purified product of the above-described polypeptide antigen contained in the dietary component. An isolated or purified polypeptide antigen is produced by, for example, (a) preparing from a tissue or cell of the dietary component using a publicly known method or a method based thereon, (b) chemical synthesis by a publicly known method of peptide synthesis using a peptide synthesizer and the like, (c) culturing a transformant comprising a DNA that encodes the polypeptide antigen, or (d) biochemical synthesis with a nucleic acid that encodes the polypeptide antigen as the template, using a cell-free transcription/translation system.

(a) When the polypeptide antigen is prepared from a tissue or cell of a dietary component, the tissue or cell is homogenized, after which a crude fraction (e.g., membrane fraction, soluble fraction) can be used as the polypeptide antigen as it is. Alternatively, extraction is performed with an acid, surfactant, alcohol or the like, and the extract can be purified and isolated by a combination of salting-out, dialysis, gel filtration, and chromatographies such as reversed phase chromatography, ion exchange chromatography, and affinity chromatography. The protein obtained may be used as the polypeptide antigen as it is, or a partial peptide may be prepared by limiting degradation using peptidase and the like and used as the polypeptide antigen.
(b) When the polypeptide antigen is prepared chemically, useful synthetic peptides include, for example, a peptide having the same structure as a protein purified from a natural material using the method described in (a) above, specifically a peptide comprising one kind or two kinds or more of the same amino acid sequence as the amino acid sequence consisting of at least three or more, preferably six or more, amino acids, at an optionally chosen site in the amino acid sequence of the protein, and the like.
(c) When a polypeptide antigen is produced using a transformant harboring a DNA, the DNA can be prepared according to a publicly known cloning method (for example, the method described in Molecular Cloning 2nd ed. (J. Sambrook et al., Cold Spring Harbor Lab. Press, 1989) and the like). Such cloning methods include (1) a method wherein a DNA that encodes a polypeptide antigen is isolated from a cDNA library of dietary components by a hybridization method using a DNA probe designed on the basis of the gene sequence that encodes the polypeptide antigen, (2) a method wherein a DNA that encodes a polypeptide antigen is prepared by a PCR method with a cDNA derived from a dietary component as the template, using DNA primers designed on the basis of the gene sequence that encodes the antigen, and the DNA is inserted into an expression vector suitable for the host, and the like. By culturing a transformant obtained by transforming a host with the expression vector in an appropriate medium, the desired polypeptide antigen can be obtained.
(d) When a cell-free transcription/translation system is utilized, useful methods include a method wherein an mRNA is synthesized with an expression vector incorporating a DNA that encodes a polypeptide antigen prepared in the same manner as (c) above (for example, an expression vector wherein the DNA is placed under the control of the T7 or SP6 promoter or the like, and the like) as the template, using a transcription reaction liquid comprising an RNA polymerase and substrate (NTPs) suitable for the promoter, after which a translation reaction is carried out with the mRNA as the template, using a publicly known cell-free translation system (e.g., Escherichia coli, rabbit reticulocytes, extract of wheat germ and the like) and the like. By adjusting the salt concentration and the like as appropriate, the transcription reaction and the translation reaction can be carried out in a lump in the same reaction liquid.

“Isolated or purified” means that an operation for removing components other than the component of interest has been performed. The content amount of the polypeptide of interest contained in the “isolated or purified polypeptide antigen” is normally 60% by weight or more, preferably 80% by weight or more, more preferably 90% by weight or more, most preferably 95% by weight or more, relative to all polypeptides in the sample.

Useful polypeptide antigens include a complete protein molecule and an antigenic peptide comprising a partial amino acid sequence thereof (partial peptide possessing antigenicity). Partial amino acid sequences include, for example, those consisting of three or more continuous amino acid residues, preferably those consisting of four or more, more preferably five or more, still more preferably six or more, continuous amino acid residues. A portion (e.g., one to several residues) of these amino acid residues may be substituted by possibly substituting groups (e.g., Cys, hydroxyl group and the like). The peptide used as the polypeptide antigen has an amino acid sequence comprising one to several such partial amino acid sequences.

In a preferred mode of embodiment, a dietary component preparation is immobilized onto an appropriate solid phase (for example, multiwell plate for immunoassay and the like). The immobilization can be achieved by adding the preparation, diluted with an ordinary buffer solution for coating as required, to the solid phase, and incubating the preparation for a given time. After the liquid is removed from the solid phase, the specimen is added to the solid phase to form an antigen-antibody complex and capture the antibody of interest on the solid phase.

Substances that react with an anti-dietary-component antibody include, for example, anti-immunoglobulin antibodies, is protein A, protein G, jacalin and the like, with preference given to anti-immunoglobulin antibodies, particularly anti-IgG antibodies or anti-IgA antibodies.

Labeling enzymes include, but are not limited to, peroxidase, β-galactosidase, alkaline phosphatase, microperoxidase, carboxypeptidase, phosphorylase and the like.

An enzyme substrate is chosen as appropriate according to the kind of labeling enzyme used; when using peroxidase as the labeling enzyme, 3,3′,5,5′-tetramethylbenzidine (TMB; Vector Laboratories Inc. Company, #SK-4400), for example, can be used.

By removing the reaction liquid, and measuring absorbance at a measuring wavelength of 450 nm on the solid phase using a microplate reader and the like, the amount of label, that is, the amount of antibody, bound to the solid phase can be measured.

Whether the anti-dietary-component antibody examined in the subject of diagnosis is elevated can be determined on the basis of, for example, the presence or absence of a statistically significant difference compared with the antibody titer in healthy animals. For example, the mean+SD (standard deviation), mean+2SD, mean+3SD and the like of measured values in healthy animals can be used as cutoff values for positivity, but these are not to be construed as limiting.

In a preferred mode of embodiment of the diagnostic method of the present invention, two kinds or more (e.g., 2, 3, 4, 5, 6, 8, 10 or 15 kinds) of antibodies out of the aforementioned 19 kinds of Crohn's disease-specific anti-dietary-component antibodies are the analytes. By combining two kinds or more of antibodies, the diagnostic sensitivity and/or diagnostic specificity for Crohn's disease can be further improved. When two kinds or more of antibodies are combined, it is preferable that an antibody against at least one kind of dietary component selected from among grapefruit, cabbage, lettuce, oat, pecan, yeast, cane sugar, celery, buckwheat and corn be contained, and it is more preferable that antibodies against at least yeast and corn be contained. In another preferred embodiment, the diagnostic method of the present invention involves the use of three kinds or four kinds or more of antibodies in combination. When three kinds or more of antibodies are combined, it is more preferable that antibodies against at least yeast, corn, and buckwheat or celery be contained. Specific examples of combinations include the following combinations of antibodies against dietary components.

### (Examples of Combinations of Two Kinds of Dietary Components)

Corn, cabbage

Corn, lettuce

Corn, buckwheat

Yeast, grapefruit

Yeast, cabbage

Yeast, celery

Yeast, lettuce

Yeast, buckwheat

Yeast, corn

Yeast, oat

Yeast, pecan

Cane sugar, cabbage

Cane sugar, corn

Cane sugar, yeast

### (Examples of Combinations of Three Kinds of Dietary Components)

Yeast, corn, buckwheat

Yeast, corn, celery

### (Examples of Combinations of Four Kinds of Dietary Components)

Yeast, corn, buckwheat, grapefruit

Yeast, corn, buckwheat, cabbage

Yeast, corn, buckwheat, green pepper

Yeast, corn, buckwheat, tomato

Yeast, corn, buckwheat, soy

Yeast, corn, buckwheat, celery

Yeast, corn, alfalfa, celery

Yeast, corn, cane sugar, celery

### (Examples of Combinations of Five Kinds of Dietary Components)

Yeast, corn, buckwheat, grapefruit, alfalfa

Yeast, corn, buckwheat, grapefruit, cane sugar

Yeast, corn, buckwheat, celery, alfalfa

Yeast, corn, buckwheat, celery, cane sugar

Yeast, corn, buckwheat, green pepper, alfalfa

Yeast, corn, buckwheat, green pepper, cane sugar

Yeast, corn, buckwheat, tomato, alfalfa

Yeast, corn, buckwheat, tomato, cane sugar

Yeast, corn, buckwheat, soy, alfalfa

Yeast, corn, buckwheat, soy, cane sugar

When a diagnosis is made using two kinds or more of anti-dietary-component antibodies in combination, results can occur in which the reaction is positive only for some antibodies and negative for the other antibodies; in such cases, preferably, the subject of diagnosis is judged to be suffering, or likely to contract, Crohn's disease, provided that the reaction is positive for any one antibody. By doing so, the diagnostic sensitivity can be dramatically improved, while maintaining a given level of diagnostic specificity.

The present invention also provides a reagent for diagnosis of Crohn's disease comprising one kind or more of dietary component preparations that react with an anti-dietary-component antibody of the present invention. Specifically, the dietary component preparation is prepared by processing a dietary component selected from among 19 kinds: grapefruit, alfalfa, avocado, cabbage, green pepper, lettuce, onion, potato (white), spinach, tomato, oat, pecan, yeast, cane sugar, celery, buckwheat, corn, rice and soy. The method of preparing the preparation is as described with respect to the method of the present invention described above. The dietary component preparation obtained can, for example, be provided in solution in water, a buffer solution, glycerol and the like in a container such as a plastic tube, and stored under refrigeration or freezing until just before use.

As stated above, in the diagnosis of Crohn's disease according to the present invention, it is preferable that two kinds or more of anti-dietary-component antibodies in the specimen be measured. Therefore, in a preferred mode of embodiment, the reagent for diagnosis of Crohn's disease of the present invention may comprise two kinds or more (e.g., 2, 3, 4, 5, 6, 8, 10 or 15 kinds) of dietary component preparations in the configuration. Preferable combinations of two kinds or more of dietary components are as described above.

When a plurality of dietary component preparations are used, they may be mixed together to make a single reagent, or may be prepared as separate reagents; however, because it is uneasy to provide substances that react specifically with respective anti-dietary-component antibodies, it is normally advantageous to prepare the preparations as separate reagents.

The present invention also provides a kit for diagnosis of Crohn's disease comprising the reagent of the present invention. The kit of the present invention comprises a dietary component preparation alone or two kinds or more thereof in combination, and can optionally comprise other components that are reagents required depending on the choice of immunological assay method and the means of detection adopted. Preferably, the kit of the present invention can comprise a substance that reacts with an anti-dietary-component antibody (for example, secondary antibodies such as anti-IgG antibody and anti-IgA antibody). The substance may be previously labeled with a labeling substance such as an enzyme, a fluorescent substance, a luminescent substance, a radioactive substance, or a coloring substance, and a labeling substance may be included in the configuration of the kit. The dietary component preparation may be immobilized on a solid phase in advance, and the solid phase may be separately contained in the configuration of the kit.

The kit of the present invention may also comprise a substrate according to the labeling substance, or a detection reagent for detecting the reaction between the labeling substance and the substrate, and may further comprise an appropriate specimen diluent, secondary antibody diluent, standard antibody, buffer solution, washing liquid, enzyme substrate liquid, reaction stopper liquid and the like for the sake of convenience in conducting the measurement. Additionally, a standard serum from a healthy person for use as a control may be contained.

The present invention is hereinafter described in more detail by means of the following Examples, by which, however, the invention is not limited in any way.

### EXAMPLES

### Reference Example 1

### Acquisition of Human Serum or Plasma

Blood was drawn from 80 Crohn's disease patients, 44 ulcerative colitis patients and 52 healthy volunteers after informed consent was obtained in writing at Social Insurance Chuo General Hospital or Ajinomoto Co., Inc., and serum or plasma was obtained. The specimens were identified by assigned numbers and the like and anonymized.

### Example 1

### Measurement of Antibody Titer (IgG) of 88 Dietary Items

After being drawn, blood was immediately centrifuged; the serum acquired was stored under freezing and sent, via Detox Inc., to Genova Diagnostics (IL, USA), to which IgG Food Antibody Assessment (88 dietary items) measurements were requested. Breakdown of the 88 dietary items was 7 items of dairy products (casein, cheddar cheese, cottage cheese, cow's milk, goat's milk, lactalbumin, yogurt), 22 items of vegetables (alfalfa, asparagus, avocado, beet, broccoli, cabbage, carrot, celery, cucumber, garlic, green pepper, lettuce, mushroom, olive, onion, pea, potato (sweet), potato (white), spinach, string bean, tomato, zucchini), 16 items of fruits (apple, apricot, banana, blueberry, cranberry, grape, grapefruit, lemon, orange, papaya, peach, pear, pineapple, plum, raspberry, strawberry), 19 items of fish/meat (clam, cod, crab, lobster, oyster, red snapper, salmon, sardine, shrimp, sole, trout, tuna, beef, chicken, egg white, egg yolk, lamb, pork, turkey), 19 items of cereals/nuts (almond, buckwheat, corn, corn gluten, gluten, kidney bean, lentil, lima bean, oat, peanut, pecan, pinto bean, rice, rye, sesame, soy, sunflower seed, walnut, wheat), and 5 other items (yeast, cane sugar, chocolate, coffee, honey). IgG was measured by ELISA; with the “mean value+2SD” of each dietary item in healthy volunteers as the cutoff, items exceeding this level were judged to be positive. The mean number of items with positive serum antibody titers out of the 88 dietary items was 11 items for the Crohn's disease patients, 2 items for the ulcerative colitis patients, and 1 item for the healthy volunteers.

The antibody positive rate (number of positive specimens/total number of specimens×100) for each item was calculated separately for the Crohn's disease patients, the ulcerative colitis patients, and the healthy volunteers.

Next, the positive rate, specificity (number of negative specimens/total number of specimens×100), and diagnosis efficiency (positive rate×specificity/100) for combinations of two to five items were calculated.

### Results

Dietary items showing significantly higher positive rates in the Crohn's disease patients than in the healthy volunteers and the ulcerative colitis patients were 19 items (grapefruit, alfalfa, avocado, cabbage, celery, green pepper, lettuce, onion, potato (white), spinach, tomato, buckwheat, corn, oat, pecan, rice, soy, yeast, cane sugar). The positive rates for the 19 items in the Crohn's disease patients (CD), the ulcerative colitis patients (UC), and the healthy volunteers (HC) are shown in Table 1.

TABLE 1

| | Positive rate (%) | | |
|---|---|---|---|
| Dietary component | CD | UC | HC |
| Grapefruit | 33 | 0 | 0 |
| Alfalfa | 24 | 0 | 0 |
| Avocado | 16 | 0 | 0 |
| Cabbage | 50 | 0 | 6 |
| Celery | 45 | 2 | 0 |
| Green pepper | 21 | 2 | 2 |
| Lettuce | 44 | 2 | 2 |
| Onion | 19 | 0 | 2 |
| Potato (white) | 16 | 0 | 0 |
| Spinach | 19 | 0 | 0 |
| Tomato | 21 | 0 | 0 |
| Buckwheat | 40 | 0 | 2 |
| Corn | 65 | 7 | 2 |
| Oat | 41 | 0 | 2 |
| Pecan | 35 | 5 | 0 |
| Rice | 30 | 2 | 0 |
| Soy | 18 | 2 | 0 |
| Yeast | 56 | 0 | 2 |
| Cane sugar | 61 | 2 | 0 |

The results of calculations of the sensitivity (the number of specimens judged to be positive relative to all specimens from Crohn's disease patients; positive rate) from the absorbance (amount of antibody) obtained by each measurement for combinations of 2 kinds of antibodies against the 19 kinds of dietary antigens are shown in Table 2.

TABLE 2
Positive rates in 80 Crohn's disease patients

| | | | | | | Green | | | Potato | |
|---|---|---|---|---|---|---|---|---|---|---|
| | Grapefruit | Alfalfa | Avocado | Cabbage | Celery | pepper | Lettuce | Onion | (white) | Spinach |
| Grapefruit | 33 | 40 | 36 | 56 | 50 | 36 | 51 | 35 | 39 | 38 |
| Alfalfa | | 24 | 29 | 56 | 49 | 33 | 50 | 31 | 31 | 30 |
| Avocado | | | 16 | 54 | 45 | 26 | 44 | 23 | 24 | 23 |
| Cabbage | | | | 50 | 61 | 51 | 60 | 54 | 54 | 55 |
| Celery | | | | | 45 | 46 | 51 | 45 | 46 | 45 |
| Green | | | | | | 21 | 46 | 30 | 29 | 30 |
| pepper | | | | | | | | | | |
| Lettuce | | | | | | | 44 | 44 | 45 | 45 |
| Onion | | | | | | | | 19 | 25 | 23 |
| Potato | | | | | | | | | 16 | 24 |
| (white) | | | | | | | | | | |
| Spinach | | | | | | | | | | 19 |
| Tomato | | | | | | | | | | |
| Buckwheat | | | | | | | | | | |
| Corn | | | | | | | | | | |
| Oat | | | | | | | | | | |
| Pecan | | | | | | | | | | |
| Rice | | | | | | | | | | |
| Soy | | | | | | | | | | |
| Yeast | | | | | | | | | | |
| Cane sugar | | | | | | | | | | |

| | | Buck- | | | | | | | Cane |
|---|---|---|---|---|---|---|---|---|---|
| | Tomato | wheat | Corn | Oat | Pecan | rice | Soy | Yeast | sugar |
| Grapefruit | 41 | 49 | 66 | 53 | 45 | 46 | 36 | 70 | 66 |
| Alfalfa | 34 | 48 | 66 | 48 | 41 | 38 | 33 | 61 | 63 |
| Avocado | 26 | 43 | 65 | 44 | 36 | 34 | 26 | 60 | 61 |
| Cabbage | 56 | 60 | 70 | 59 | 59 | 58 | 54 | 75 | 70 |
| Celery | 49 | 51 | 69 | 54 | 49 | 48 | 50 | 76 | 68 |
| Green | 29 | 45 | 66 | 46 | 39 | 39 | 28 | 63 | 65 |
| pepper | | | | | | | | | |
| Lettuce | 49 | 51 | 70 | 55 | 46 | 49 | 50 | 74 | 68 |
| Onion | 29 | 44 | 65 | 45 | 39 | 34 | 29 | 63 | 63 |
| Potato | 29 | 41 | 65 | 44 | 36 | 34 | 26 | 63 | 63 |
| (white) | | | | | | | | | |
| Spinach | 28 | 45 | 66 | 45 | 39 | 33 | 28 | 60 | 64 |
| Tomato | 21 | 48 | 68 | 49 | 41 | 36 | 30 | 61 | 66 |
| Buckwheat | | 40 | 70 | 53 | 43 | 46 | 46 | 73 | 66 |
| Corn | | | 65 | 66 | 66 | 65 | 68 | 84 | 74 |
| Oat | | | | 41 | 50 | 44 | 49 | 73 | 68 |
| Pecan | | | | | 35 | 41 | 41 | 70 | 64 |
| Rice | | | | | | 30 | 38 | 65 | 65 |
| Soy | | | | | | | 18 | 64 | 66 |
| Yeast | | | | | | | | 56 | 80 |
| Cane sugar | | | | | | | | | 61 |

For each of the antibodies against the individual dietary antigens alone and combinations of 2 kinds to 19 kinds thereof, sensitivity and specificity were calculated from the absorbance (amount of antibody) obtained by each measurement. For some combinations of 2 items to 5 items, positive rates for CD, specificities for UC and HC, and diagnosis efficiencies for CD relative to UC are shown in Table 3-1 to Table 3-4.

TABLE 3-1

| | | | |
|---|---|---|---|
| | Positive | | CD diagnosis |
| Combination | rate (%) | Specificity (%) | efficiency (%) |

| of 2 items | CD | UC | HC | Relative to UC |
|---|---|---|---|---|
| Yeast corn | 84 | 93 | 96 | 78.1 |
| Yeast cane | 80 | 98 | 98 | 78.4 |
| sugar | | | | |
| Yeast | 76 | 98 | 98 | 74.5 |
| celery | | | | |
| Yeast | 70 | 93 | 96 | 65.1 |
| buckwheat | | | | |
| Corn | 73 | 100 | 96 | 73.0 |
| buckwheat | | | | |

TABLE 3-2

| | | | |
|---|---|---|---|
| | Positive | | CD diagnosis |
| Combination | rate (%) | Specificity (%) | efficiency (%) |

| of 3 items | CD | UC | HC | Relative to UC |
|---|---|---|---|---|
| Yeast corn | 86 | 93 | 96 | 80.0 |
| celery | | | | |
| Yeast corn | 86 | 93 | 94 | 80.0 |
| buckwheat | | | | |

TABLE 3-3

| | Positive | Specificity | | CD diagnosis |
|---|---|---|---|---|
| | rate (%) | (%) | | efficiency (%) |
| Combination of 4 items | CD | UC | HC | Relative to UC |
| Yeast corn buckwheat | 88 | 93 | 94 | 81.8 |
| grapefruit | | | | |
| Yeast corn buckwheat | 88 | 93 | 90 | 81.8 |
| cabbage | | | | |
| Yeast corn buckwheat | 88 | 93 | 92 | 81.8 |
| green pepper | | | | |
| Yeast corn buckwheat | 88 | 93 | 94 | 81.8 |
| tomato | | | | |
| Yeast corn buckwheat | 88 | 91 | 94 | 80.1 |
| soy | | | | |
| Yeast corn buckwheat | 88 | 93 | 94 | 81.8 |
| celery | | | | |
| Yeast corn alfalfa | 88 | 93 | 96 | 81.8 |
| celery | | | | |
| Yeast corn cane | 88 | 91 | 96 | 80.1 |
| sugar celery | | | | |

TABLE 3-4

| | Positive | Specificity | | CD diagnosis |
|---|---|---|---|---|
| | rate (%) | (%) | | efficiency (%) |
| Combination of 5 items | CD | UC | HC | Relative to UC |
| Yeast corn buckwheat | 89 | 93 | 94 | 82.8 |
| grapefruit alfalfa | | | | |
| Yeast corn buckwheat | 89 | 91 | 94 | 81.0 |
| grapefruit cane sugar | | | | |
| Yeast corn buckwheat | 89 | 93 | 94 | 82.8 |
| celery alfalfa | | | | |
| Yeast corn buckwheat | 89 | 91 | 94 | 81.0 |
| celery cane sugar | | | | |
| Yeast corn buckwheat | 89 | 93 | 92 | 82.8 |
| green pepper alfalfa | | | | |
| Yeast corn buckwheat | 89 | 91 | 92 | 81.0 |
| green pepper cane | | | | |
| sugar | | | | |
| Yeast corn buckwheat | 89 | 93 | 94 | 82.8 |
| tomato alfalfa | | | | |
| Yeast corn buckwheat | 89 | 91 | 94 | 81.0 |
| tomato cane sugar | | | | |
| Yeast corn buckwheat | 89 | 91 | 94 | 81.0 |
| soy alfalfa | | | | |
| Yeast corn buckwheat | 89 | 89 | 94 | 79.2 |
| soy cane sugar | | | | |

The results above demonstrated that by combining antibodies, the sensitivity is significantly improved compared with the amount of one kind of antibody measured alone. For all combinations, high specificity was observed, the diagnosis efficiency as calculated by multiplying the sensitivity and the specificity likewise improved remarkably when antibodies were combined.

Therefore, according to the present invention, specific and efficient diagnosis of Crohn's disease is possible, which in turn makes it is possible to diagnose Crohn's disease safely and highly sensitively, without undergoing invasive methods that require high levels of experience and skills and pose physical and mental sufferings on the patient, such as endoscopic examination.

### Example 2

### Measurement of Serum Antibody Titers Against Dietary Component Preparations

Of the dietary components shown by the above-described results to be useful, corn, yeast, buckwheat, and celery were selected; for sera from 98 CD, 50 UC, and 52 HC, serum antibody titers against various dietary component preparations were measured. In this Example, powder materials were used as the dietary component preparations.

As the antigen liquids for measuring serum antibody titers, supernatants obtained by centrifuging (5000 rpm, 5 minutes) suspensions of various powders of corn, yeast, buckwheat, and celery (all manufactured by Allergon Company) in PBS(−) were used. Each antigen liquid was prepared using a coating buffer (manufactured by SIGMA Company, cat. No. 076K8206) to obtain a protein concentration of 1 μg/ml each and added to an ELISA plate (manufactured by Sumitomo Bakelite Company, cat. No. MS-8896F) at 50 μl/well, and a reaction was allowed to proceed at 4° C. overnight. After the antigen liquid in the wells was removed, the plate was once washed with a washing liquid (Immunoblock (manufactured by DS Pharma Biomedical Company, cat. No. KN001A), diluted 20 fold with PBS(−) containing 0.1% Tween 20), and reacted with Immunoblock, diluted 5 fold with distilled water, at room temperature for 1 hour. After the plate was once washed with the washing liquid, each serum, diluted 10000 fold with the washing liquid, was added, and a reaction was allowed to proceed at room temperature for 2 hours. After the reaction, the plate was washed with the washing liquid 3 times, and an HRP-labeled mouse anti-human IgG monoclonal antibody (manufactured by Invitrogen Company, cat. No. 05-4220), diluted 500 fold with the washing liquid, was added; a reaction was allowed to proceed at room temperature for 1 hour. Subsequently, the plate was washed with the washing liquid 3 times and reacted with TMB (manufactured by BD Biosciences Company, cat. No. 555214); the reaction was stopped with 2 N sulfuric acid, and absorbance at a wavelength of 450 nm was measured using a microplate reader (BIO-RAD Benchmark Plus).

An anti-human IgG antibody (manufactured by EXBIO Company, cat. No. 11-31-9-C100), diluted 1000 fold with the coating buffer, was added to the same plate at 50 μl/well and reacted with a reference serum (manufactured by BETHYL Company, cat. No. RS10-101) at 0.01 to 10 μg/ml, and absorbance at a wavelength of 450 nm were measured at each concentration; a working curve was generated, and the IgG Titer of the serum sample was calculated.

The “mean value+2SD” of IgG Titers against each dietary component in the healthy volunteers was calculated; when this level was exceeded, the sample was judged to be positive. The positive rate (number of positive specimens/total number of specimens×100) for each dietary component was calculated for each of CD, UC, and HC.

### Results

The positive rates for the various dietary components are shown in Table 4. Despite the use of the different dietary materials, the antibody positive rates measured using the dietary component preparations were nearly equivalent to the positive rates measured at Genova Diagnostics for all of CD, UC, and HC (Table 1).

TABLE 4

| | Positive rate (%) | | |
|---|---|---|---|
| Dietary component | CD | UC | HC |
| Corn | 63 | 2 | 2 |
| Buckwheat | 32 | 0 | 2 |
| Celery | 46 | 0 | 6 |
| Yeast | 34 | 2 | 4 |

Furthermore, the positive rates for CD, specificities for UC and HC, and diagnosis efficiencies for CD relative to UC for combinations of two to four items out of the aforementioned four items of dietary components are shown in Tables 5-1 to 5-3. When measurements were taken using the dietary component preparations, the positive rates were about 70% and the specificities were 90% or more for all combinations of two to four items; diagnosis efficiencies of about 65 to 74% were obtained. These results revealed slightly lower positive rates but equivalent or higher specificities compared with the results measured at Genova Diagnostics (Tables 3-1 to 3-3). These results suggested that Crohn's disease can be diagnosed using this assay method.

TABLE 5-1

| | | Positive | | | CD diagnosis |
|---|---|---|---|---|---|
| Combination of | | rate (%) | Specificity (%) | | efficiency (%) |
| 2 items | | CD | UC | HC | Relative to UC |
| Yeast | Corn | 70 | 96 | 94 | 67.2 |
| Corn | Buckwheat | 66 | 98 | 98 | 64.7 |

TABLE 5-2

| | | | Positive | Specific- | | CD diagnosis |
|---|---|---|---|---|---|---|
| Combination of 3 | | | rate (%) | ity (%) | | efficiency (%) |
| items | | | CD | UC | HC | Relative to UC |
| Yeast | Corn | Celery | 74 | 96 | 90 | 71.0 |
| Yeast | Corn | Buckwheat | 73 | 96 | 94 | 70.1 |

TABLE 5-3

| | | | | Positive | Specific- | | CD diagnosis |
|---|---|---|---|---|---|---|---|
| Combination of 4 | | | | rate (%) | ity (%) | | efficiency (%) |
| items | | | | CD | UC | HC | Relative to UC |
| Yeast | Corn | Celery | Buck- | 77 | 96 | 90 | 73.9 |
| | | | wheat | | | | |

### Example 3

### Identification of Antigens

For the dietary antigens shown by the results of Example 1 to be useful, proteins that can be specifically used as antigens were identified.

Out thereof, rice powder (manufactured by Allergon Company) was suspended in 2% SDS solution, and a suspension of the powder equivalent to 125 μg per lane was subjected to SDS-PAGE. After separation by SDS-PAGE, each protein was transferred to a PVDF membrane under 60 mA constant amperage conditions for 60 minutes. The PVDF membrane was stained with Rapid Stain CBB (manufactured by Nacalai Tesque Company) to detect the protein, after which Western blotting was performed with an HC serum or a CD serum that was positive for the antibody titer against rice, each diluted 1000 fold, as the primary antibody, and with an HRP-labeled anti-human IgG antibody (manufactured by GE Healthcare Company), diluted 5000 fold, as the secondary antibody. The bands specifically detected with the CD serum were subjected to Peptide Mass Finger printing (PMF) analysis via a protein research network (http://protein-research.org/).

### Results

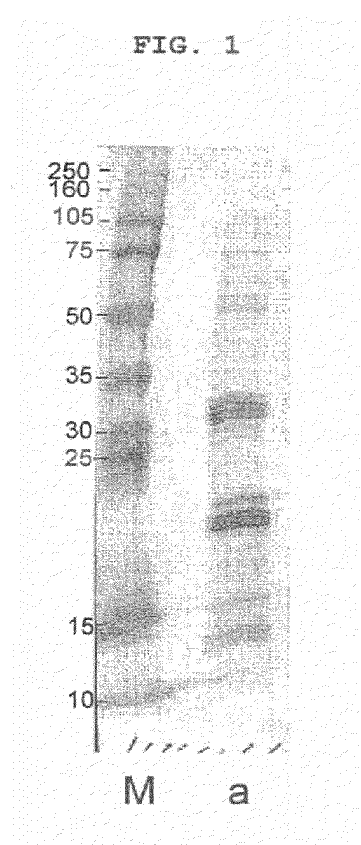

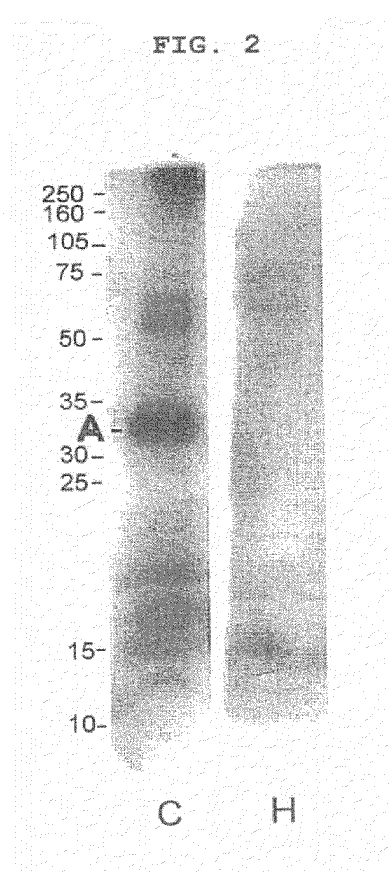

The results of the CBB staining of rice proteins are shown in FIG. 1, and the results of the Western blotting are shown in FIG. 2. The protein of the band A in FIG. 2 reacted specifically with the antibody in the CD serum. The results of the PMF analysis of the protein of the band A (about 33 kDa) are shown in Table 6. From the molecular weight patterns of peptide fragments obtained by the PMF analysis, the protein of the band A was estimated to be Glutelin (Accession No. ABF96730.1) (Table 7).

TABLE 6

| Dietary | | |
|---|---|---|
| component | Protein | Accession NO. |
| Rice | glutelin | ABL74547.1 |
| | | ABL74544.1 |
| | | ABF96730 |
| | glyoxylase1 | BAD05593.1 |
| | enolase | AAP94211.1 |
| | UDP-glucose pyrophosphorylase | ABD57308.1 |
| | asparatic protease | EAZ12992.1 |
| | | BAA06876.1 |
| | prolamin | AAA50319 |
| | oleosin | AAC02239.1 |
| Buckwheat | clathrin | ABA95598.1 |
| | | CAN79917.1 |
| Corn | 2,3-bisphosphoglycerate- | NP001105584.1 |
| | independent phosphoglycerate | |
| | protein disulfide isomerase | NP001105754.1 |
| | ketol-acid reductoisomerase | ACG35752.1 |
| | elongation factor 1 alpha | NP001105933.1 |
| | phenylalanine ammonia-lyase | NP001105334.1 |
| Cane sugar | triphosphate isomerase 1 | AAB81110 |
| | NBS-LRR type RGA | AAZ99763 |

TABLE 7

| peptide | molecular weight |
|---|---|
| AP-1 | 931.457 |
| AP-2 | 959.467 |
| AP-3 | 1123.639 |
| AP-4 | 1307.635 |
| AP-5 | 1629.775 |
| AP-6 | 1669.766 |
| AP-7 | 1702.885 |
| AP-8 | 1840.929 |
| AP-9 | 1914.925 |
| AP-10 | 1921.040 |
| AP-11 | 2282.947 |
| AP-12 | 2559.215 |
| AP-13 | 3339.559 |
| AP-14 | 3426.586 |
| AP-15 | 4106.025 |
| AP-16 | 4193.053 |

Also, proteins other than Glutelin in the rice powder were identified by the same method as the above; furthermore, using powders of buckwheat, corn and cane sugar (all manufactured by Allergon Company) as dietary components other than rice, useful protein antigens contained in the various dietary components were identified by the same procedures as the above for the rice powder. The results are shown in Table 8.

TABLE 8

| | molecular weight | | | | | |
|---|---|---|---|---|---|---|
| peptide | observed | theoretical | delta | corresponding sequence | Head | Tail |
| AP-2 | 959.467 | 959.47 | −0.003 | [K]FRDEHQK[I] | 147 | 153 |
| AP-8 | 1840.929 | 1840.93 | −0.001 | [K]IGQQLYRYEARDNSK[N] | 211 | 225 |

### Example 4

### Measurement of Serum Antibody Titers Using Glutelin

Total RNA was extracted from rice (Hitomebore) using the Spectrum (trademark) Plant Total RNA kit (manufactured by SIGMA Aldrich Company). The full-length Glutelin gene was acquired using the following primers designed from the mRNA sequence (Accession No. NM_{—}001056948) of Glutelin type-A 3 precursor (Accession No. ABF96730.1).

| Forward primer: | (SEQ ID NO: 1) |
|---|---|
| GGATCCATGGCAACCATCAAATTCCCTATAG | |
| Reverse primer: | (SEQ ID NO: 2) |

GCGGCCGCTTAGTGGTGATGATGGTGATGTGCACTC

The acquired Glultelin gene was introduced into the expression vector pGEX-6P-1 (manufactured by GE Healthcare Company) to construct an His tag-GST (Glutathione S-Transferase) fusion Glutelin expression vector (pGEX-GSTOSG30His). Using Escherichia coli BL21-CodonPlus(DE3)-RIPL (manufactured by Stratagene Company) incorporating pGEX-GSTOSG30His, the GST-Glutelin-His tag fusion protein was expressed; the protein was purified from the cell bodies using Ni-NTA Agarose (manufactured by QIAGEN Company) and subjected to the anti-Glutelin antibody titer assay described below.

Sera from 98 CD patients and 52 HC patients were assayed to determine anti-Glutelin antibody titers by ELISA. The anti-Glutelin antibody titers were calculated by subtracting the value of the reaction to GST from the value obtained as the reaction to GST fusion protein (Glutelin-GST) according to the method of Sutton C L et al. (Gastroenterology 119:23-31, 2000).

Antigen liquids were prepared at 100 μg/ml and 32 μg/ml to reach an equimolar ratio of Glutelin-GST and GST using a coating buffer (manufactured by SIGMA Company, cat. No. 076K8206); each antigen liquid was added to an ELISA plate (manufactured by Sumitomo Bakelite Company, cat. No. MS-8896F) at 50 μl/well, and a reaction was allowed to proceed at 4° C. overnight. After the antigen liquid was removed, the plate was once washed with a washing liquid (Immunoblock (manufactured by DS Pharma Biomedical Company, cat. No. KN001A), diluted 20 fold with PBS(−) containing 0.1% Tween 20) and reacted with Immunoblock, diluted 5 fold with PBS(−) containing 0.1% Tween 20, at room temperature for 1 hour. After the plate was once washed with the washing liquid, each serum, diluted 100 fold with the washing liquid, was added to each well, and a reaction was allowed to proceed at room temperature for 2 hours. Next, the plate was washed with the washing liquid 3 times and reacted with an HRP-labeled mouse anti-human IgG monoclonal antibody (manufactured by Invitrogen Company, cat. No. 05-4220), diluted 500 fold with the washing liquid, at room temperature for 1 hour. After the reaction, the plate was washed with the washing liquid 3 times and reacted with TMB (manufactured by BD Biosciences Company, cat. No. 555214); the reaction was stopped with 2 N sulfuric acid, and absorbance at a wavelength of 450 nm was measured using a microplate reader (BIO-RAD Benchmark Plus).

An anti-human IgG antibody (manufactured by EXBIO Company, cat. No. 11-31-9-C100), diluted 1000 fold with the coating buffer, was added to the same plate at 50 μl/well and reacted with a reference serum (manufactured by BETHYL Company, cat. No. RS10-101) at 0.01 to 10 μg/ml, and absorbance at a wavelength of 450 nm was measured at each concentration; a working curve was generated, and the IgG Titer of each serum sample was calculated.

### Results

The “mean value+2SD” or more of the IgG Titers in the HC was judged to indicate a positive reaction. Since the mean value of IgG in the HC was 0.09, and also since the 2SD value was 0.56, a value of 0.64 or more was judged to indicate a positive reaction. In this evaluation, positive specimens were obtained from 29 of the 98 CD, the positive rate being 30%. In contrast, positive specimens were obtained from 2 of the 52 healthy volunteers, the positive rate being 3.8%.

The results above reveal positive rates comparable to the positive rate of 30% in the CD and the positive rate of 0% in the healthy volunteers, shown for rice in Table 1.

By using instead a powder material of a dietary component or a single antigen protein in the diet as a method for measuring antibody titers against diets as described above, it is possible to determine positivity for the antibody against the diet and diagnose Crohn's disease. By utilizing recombinant protein production, an antigen protein can be supplied homogeneously and stably; using such a single antigen protein thus supplied, it is possible to realize stable production of a diagnostic kit and an improvement of its reliability.

### INDUSTRIAL APPLICABILITY

By combining measurements of Crohn's disease-specific anti-dietary-component antibodies, it is possible to dramatically improve the efficiency and specificity of the diagnosis of Crohn's disease, and which in turn dramatically expands the range where the diagnosis of Crohn's disease can be established, without undergoing invasive methods such as endoscopy, which require high technical skills and pose major burdens on the patient.

This application is based on a patent application No. 2009-052692 filed in Japan (filing date: Mar. 5, 2009), the contents of which are incorporated in full herein.

1. A diagnostic method for Crohn's disease in a subject, comprising measuring antibodies against one kind or more of dietary components selected from the group consisting of grapefruit, alfalfa, avocado, cabbage, green pepper, lettuce, onion, potato (white), spinach, tomato, oat, pecan, yeast, cane sugar, celery, buckwheat, corn, rice and soy (with the provision that when measuring an antibody against one kind of dietary component, the dietary component is other than celery, buckwheat, corn, yeast and soy) in a specimen collected from the subject.

2. The method according to claim 1, wherein the method comprises measuring antibodies against two kinds or more of dietary components.

3. The method according to claim 2, wherein at least one kind of dietary component is other than celery, buckwheat, corn, yeast and soy.

4. The method according to claim 2 or 3, wherein the method comprises measuring an antibody against at least one kind of dietary component selected from the group consisting of grapefruit, cabbage, lettuce, oat, pecan, yeast, cane sugar, celery, buckwheat and corn.

5. The method according to any one of claims 2 to 4, wherein the method comprises measuring antibodies against at least yeast and corn.

6. The method according to any one of claims 2 to 5, wherein the method comprises measuring antibodies against three kinds or more of dietary components.

7. The method according to claim 6, wherein the method comprises measuring antibodies against at least yeast, corn, and buckwheat or celery.

8. The method according to any one of claims 1 to 7, wherein the method comprises measuring antibodies against polypeptide antigens contained in dietary components.

9. The method according to claim 8, wherein the polypeptide antigen is Glutelin.

10. A reagent for diagnosis of Crohn's disease comprising a preparation of a dietary component selected from the group consisting of grapefruit, alfalfa, avocado, cabbage, green pepper, lettuce, onion, potato (white), spinach, tomato, oat, pecan, rice and cane sugar.

11. The reagent according to claim 10, wherein the preparation is an isolated or purified polypeptide antigen.

12. The reagent according to claim 11, wherein the polypeptide antigen is Glutelin or a partial peptide thereof possessing antigenicity.

13. A kit for diagnosis of Crohn's disease comprising a preparation of two kinds or more of dietary components selected from the group consisting of grapefruit, alfalfa, avocado, cabbage, green pepper, lettuce, onion, potato (white), spinach, tomato, oat, pecan, yeast, cane sugar, celery, buckwheat, corn, rice and soy.

14. The kit according to claim 13, wherein the preparation is an isolated or purified polypeptide antigen.

15. The kit according to claim 14, wherein the polypeptide antigen is Glutelin or a partial peptide thereof possessing antigenicity.


SEQUENCE LISTING (2 sequences)

SEQ ID NO: 1 - DNA, 31 bp, Artificial Sequence
  Description of Artificial SequencePCR primer for amplifying glut elin gene
ggatccatggcaaccatcaaattccctatag

SEQ ID NO: 2 - DNA, 36 bp, Artificial Sequence
  Description of Artificial SequencePCR primer for amplifying glut elin gene
gcggccgcttagtggtgatgatggtgatgtgcactc